(19) United States
(12) Patent Application Publication
(10) Pub. No.: US 2006/0079727 A1
(43) Pub. Date: Apr. 13, 2006

(54) Short term post surgical cavity treatment devices and methods

(75) Inventors: Ary S. Chernomorsky, Walnut Creek, CA (US); James W. Vetter, Portola Valley, CA (US); Jordan S. Tuttle, Menlo Park, CA (US)

Correspondence Address: YOUNG LAW FIRM;A PROFESSIONAL CORPORATION, 4370 ALPINE ROAD SUITE 106, PORTOLA VALLEY, CA, 94028, US

(73) Assignee: Rubicor Medical, Inc., Redwood City, CA (US)

(21) Appl. No.: 10/934,191

(22) Filed: Sep. 3, 2004

(51) Int. Cl. A61M 36/00

(52) U.S. Cl. 600/3

(57) ABSTRACT

The present short-term post surgical cavity treatment device includes a first portion including an inflatable balloon at the distal end of the device and a second portion coupled to the first portion that defines at least an inflation lumen and a treatment lumen. The second portion may be configured such that the inflation lumen enables inflation of the inflatable balloon. An inflation port may be coupled to the inflation lumen to enable introduction of a fluid or gas into the inflatable balloon. The second portion of the present device may be configured such that the treatment lumen enables various short-term cavity treatments, therapies and methods within the cavity in conjunction with the inflatable balloon. The balloon may be configured so as to inflate in a predetermined shape. This predetermined shape may be configured so as to substantially match the shape of the cavity within the tissue. The inflatable balloon may also be configured, treated, coated or otherwise configured so as to carry out a beneficial function on the tissue walls defining the post surgical cavity.

This application claims the benefit under 35 U.S.C. §119(e) of Provisional Patent Application Ser. No. 60/500,069, filed Sep. 3, 2003.

### BACKGROUND OF THE INVENTION

1. Field of the Invention

The present invention pertains to the field of devices and methods for the short-term treatment of post surgical cavities in soft tissues, such as breast tissues, for example.

2. Description of the Related Art

Breast biopsies are routinely performed in the United States following a detection of abnormalities discovered through mammographic visualization, manual palpation or ultrasound examination. There are a number of traditional methods to obtain breast biopsy tissue samples, including surgical excisional biopsies and stereotactic and ultrasound guided needle breast biopsies. Recently, methodologies have emerged that are based upon percutaneous minimally invasive large intact tissue sample collection.

Commonly assigned U.S. Pat. No. 6,022,362 discloses a novel approach to soft tissue excisional devices. As disclosed therein, the excisional device includes a shaft that includes a selectively actuable cut and collect assembly near the distal tip thereof. The cut and collect assembly is configured to selectively bow away from and to retract back toward the shaft. The excisional device is, therefore, configured to cut the soft tissue and to collect the tissue severed by the cutting tool as the excisional is rotated. The use of such devices results in a unique cavity connected to the skin by a narrow access path. However, it is becoming apparent that the post-biopsy biopsy cavities left by these procedures may both offer and require different post procedural treatments, as compared to the cavities left by needle, core biopsy procedures or open surgical procedures, due to the different nature, size and shape of the cavity created by conventional biopsy devices, as well as the narrow connection to the skin characterized by percutaneous approaches. As may be appreciated from the foregoing, there has developed a need for methods and devices for the short-term treatment of such cavities.

### SUMMARY OF THE INVENTION

According to an embodiment thereof, the present invention is a method of treating a surgical cavity of a patient, the surgical cavity defining a narrow access path and a relatively larger cavity chamber. The method may include steps of providing a cavity treatment device having a balloon and a shaft coupled to the balloon, the balloon being configured to assume a collapsed un-inflated state and an inflated state in which the balloon assumes a shape that defines a narrow proximal portion and a wider distal portion; inserting the balloon into the surgical cavity while the balloon is in the collapsed un-inflated state; causing the balloon to assume its inflated state such that the narrow proximal portion substantially fills the narrow access path and the wider distal portion substantially fills the relatively larger chamber.

The providing step may be carried out with the shaft defining an inflation lumen in fluid communication with the balloon, and the causing step may include a step of introducing a gas or a fluid (or other inflation medium) into the inflation lumen to inflate the balloon. The introducing step may be carried out such that the exterior surface of the balloon presses against tissue walls of the surgical cavity so as to provide hemostatis within the surgical cavity. The providing step may be carried out with the shaft defining a treatment lumen having a distal free end configured to be in fluid communication with the surgical cavity, and the method further may include a step of introducing a therapeutic agent into the treatment lumen to deliver the therapeutic agent to the surgical cavity. The treatment lumen may include a semipermeable membrane disposed at a distal free end thereof and the method may further include a step of delivering the therapeutic agent to the surgical cavity through the semipermeable membrane. The method further may include a step of introducing a lymphatic mapping agent into the surgical cavity through the treatment lumen. The method further may include a step of introducing a radioactive substance into the surgical cavity through the treatment lumen. The method further may include a step of introducing a photodynamic substance into the surgical cavity through the treatment lumen. The method further may include a step of introducing a plurality of radioactive elements into the treatment lumen. A distal portion of the treatment lumen may be configured to bend so as to traverse an interior space defined by the balloon in the inflated state and the method further may include a step of introducing a plurality of radioactive elements into the treatment lumen to the distal portion thereof.

At least a portion of an exterior surface of the balloon may be coated with a biologically active substance that may provide, for example, an antimicrobial, antibiotic, anti-inflammatory, analgesic, steroidal and/or anti-adhesion (e.g., adhesion preventing or inhibiting) function. For example, the biologically active substance may be or include collagen, gelatin, a mucopolysaccaride and/or hyaluronic acid, for example. All or a portion of the exterior surface of the balloon may define a predetermined texture. The predetermined texture may be achieved by exposing the exterior surface to a plasma and/or a corona discharge surface treatment, for example. The exterior surface of the balloon may be chemically treated with a surfactant.

The balloon may have a first portion that is relatively less elastic than a second portion of the balloon. The first portion of the balloon may be relatively thicker than the second portion of the balloon. The balloon may assume a generally igloo (or other predetermined) shape when in its inflated state. A valve may be fitted to the inflation lumen, the valve being configured to enable the balloon to gradually deflate at a predetermined rate (over a treatment period, for example). The cavity treatment device may be removed from the patient when the balloon has deflated and assumed its deflated state. The shaft may include visible indicia that are configured to enable a physician to correctly orient the balloon within the surgical cavity during the inserting step.

According to another embodiment, the present invention is also a surgical cavity treatment device for treating a surgical cavity having a narrow access path and a relatively larger cavity chamber, the cavity treatment device comprising: a balloon configured to assume a collapsed un-inflated state and an inflated state; a shaft coupled to the balloon; and an inflation lumen that is in fluid communication with the balloon, and a treatment lumen having an opening that is configured to be in fluid communication with the surgical cavity to enable delivery of a therapeutic agent to the surgical cavity.

The surgical cavity treatment device may also include a semipermeable membrane disposed at the opening in the treatment lumen. The treatment lumen may define a proximal portion and a distal portion that is substantially co-extensive with the balloon and the distal portion may be configured to bend at an angle relative to the proximal portion so as to traverse an interior space defined by the balloon in the inflated state. At least a portion of the exterior surface of the balloon may be coated with a biologically active substance. The biologically active substance may have an antimicrobial, antibiotic, anti-inflammatory, analgesic, steroidal and/or anti-adhesion function, for example. The biologically active substance in the providing step may be or include one or more of collagen, gelatin, a mucopolysaccaride and/or hyaluronic acid, for example. At least a portion of the exterior surface of the balloon may have a predetermined texture achieved by a plasma and/or corona discharge surface treatment, for example. At least a portion of the exterior surface of the balloon may be chemically treated with a surfactant.

The balloon may have a first portion and a second portion, the first portion being relatively less elastic than the second portion. The balloon may have a first portion and a second portion, the first portion being relatively thicker than the second portion. In its inflated state, the balloon may have a generally igloo shape, for example. The device may further include a valve fitted to a proximal end of the inflation lumen, the valve being configured to enable the balloon to gradually deflate at a predetermined rate. The device may also include visible indicia on the shaft, the visible indicia being configured to enable a physician to correctly orient the balloon within the surgical cavity.

According to yet another embodiment, the present invention is also a method for carrying out brachytherapy in a post surgical cavity within a patient, the method comprising steps of: providing a cavity treatment device that includes a balloon configured to assume a collapsed state and an expanded state; a shaft coupled to the balloon; introducing the balloon of the cavity treatment device into the cavity with the balloon in its collapsed state; causing the balloon to assume its expanded state, and inserting and advancing a treatment material into the treatment lumen.

The inserting step may be carried out with the treatment material being a radioactive material. The inserting step may be carried out with the treatment material passing through a semipermeable membrane disposed at an opening in the treatment lumen. The inserting step may be carried out by introducing a lymphatic mapping agent into the surgical cavity through the treatment lumen. The inserting step may be carried out with the material being a photodynamic substance. The inserting step may also be carried out with the material being a plurality of radioactive elements. The providing step may be carried out with the treatment lumen configured to bend so as to traverse an interior space defined by the balloon in the inflated state.

### BRIEF DESCRIPTION OF THE DRAWINGS

FIG. 1 is a representation of an exemplary post surgical cavity.

FIG. 2 is a cross-sectional view of a short-term post surgical cavity treatment device, according to an embodiment of the present invention, with the inflatable balloon thereof in a non-inflated or partially inflated state.

FIG. 3 is a cross-sectional view of a short-term post surgical cavity treatment device in which the inflatable balloon thereof is in an inflated state, according to an embodiment of the present invention.

FIG. 4 is a cross-sectional view of the short-term post surgical cavity treatment device, taken along cross-sectional lines I-I′ of FIG. 3, according to an embodiment of the present invention.

FIG. 5 is a cross-sectional view of another embodiment of the present short-term post surgical cavity treatment device.

FIG. 6 is a cross-sectional view of yet another embodiment of the present short-term post surgical cavity treatment device.

FIG. 7 is a cross-sectional view of still another embodiment of the present short-term post surgical cavity treatment device.

### DESCRIPTION OF THE INVENTION

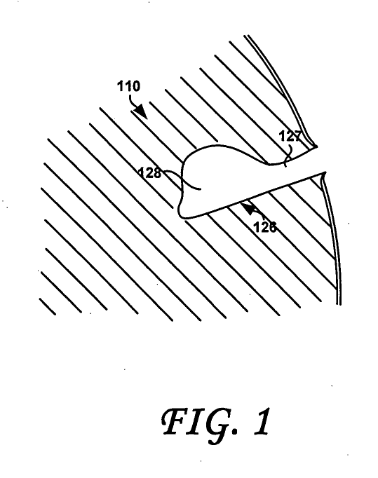

According to an embodiment thereof, the present short-term post surgical cavity treatment device includes a first portion including an inflatable balloon at the distal end of the device and a second portion coupled to the first portion that includes surfaces defining at least an inflation lumen and a treatment lumen. The second portion may be configured such that the inflation lumen enables inflation of the inflatable balloon. An inflation port may be coupled to the inflation lumen to enable introduction of a fluid or gas into the inflatable balloon. The second portion of the present device may be configured such that the treatment lumen enables various short-term cavity treatments and therapy modes and methods. The balloon may be configured so as to inflate in a predetermined shape. For example, this predetermined shape may be configured so as to substantially match the shape of the cavity within the tissue. The inflatable balloon may also be treated, coated or otherwise configured so as to carry out a beneficial function on the tissue walls defining the post surgical cavity. For example, embodiments of the present short-term post surgical cavity treatment device may be configured such that the inflatable balloon thereof assumes a shape, upon inflation, that is similar to the shape of the post surgical cavity shown in FIG. 1.

As shown in FIG. 1, the cavity 126 is formed within the tissue 110 (such as, for example, breast tissue). The exemplary cavity 126 is characterized by a relatively narrow access path 127 that emerges into a larger cavity chamber 128 formed by the extension and rotation of the cut and collect assembly during the above-described excisional procedure. After the excisional device is withdrawn from the patient, portions of the cavity 126 and/or access path 127 may settle and collapse somewhat, as the interior tissue walls defining the cavity 126 are no longer supported by the tissue previously occupying that space.

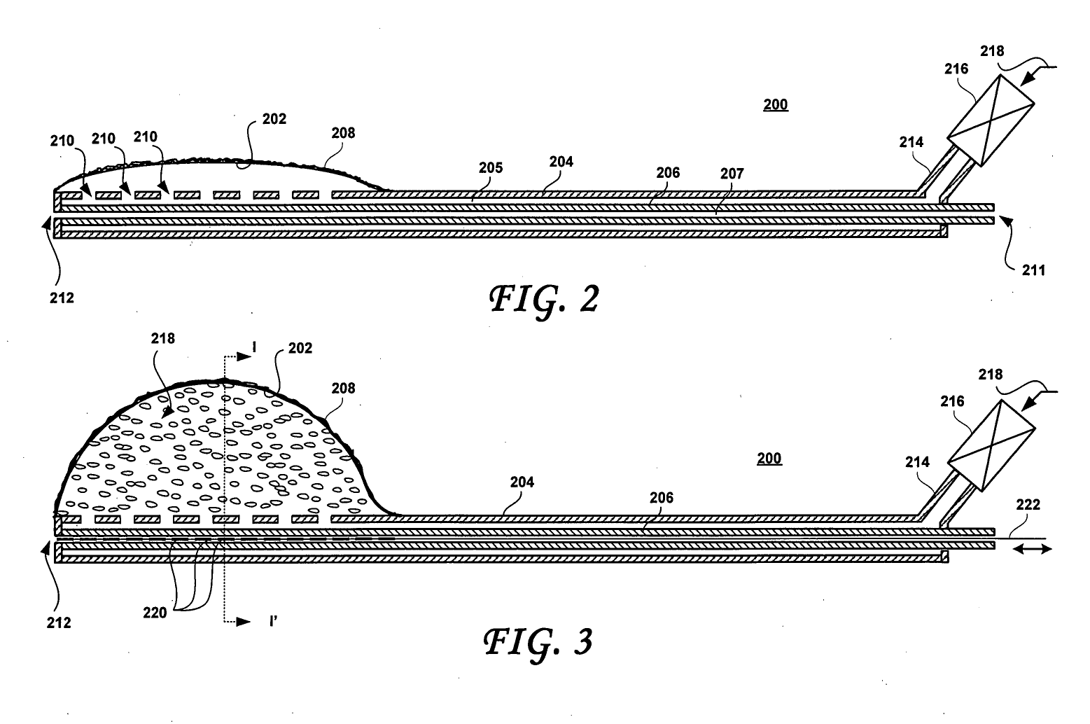

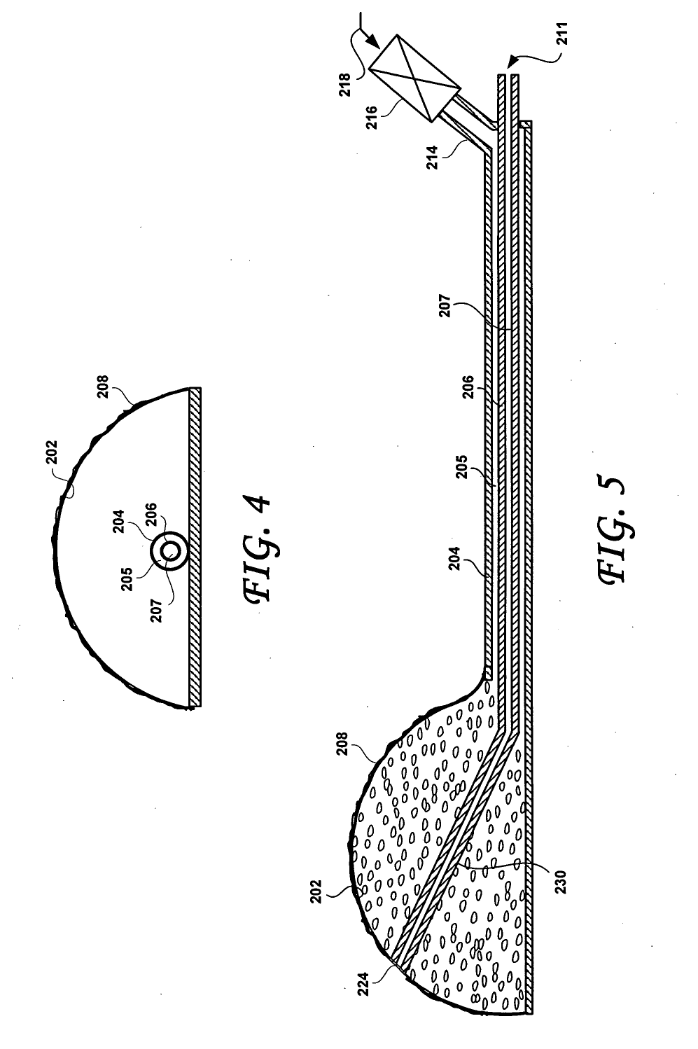

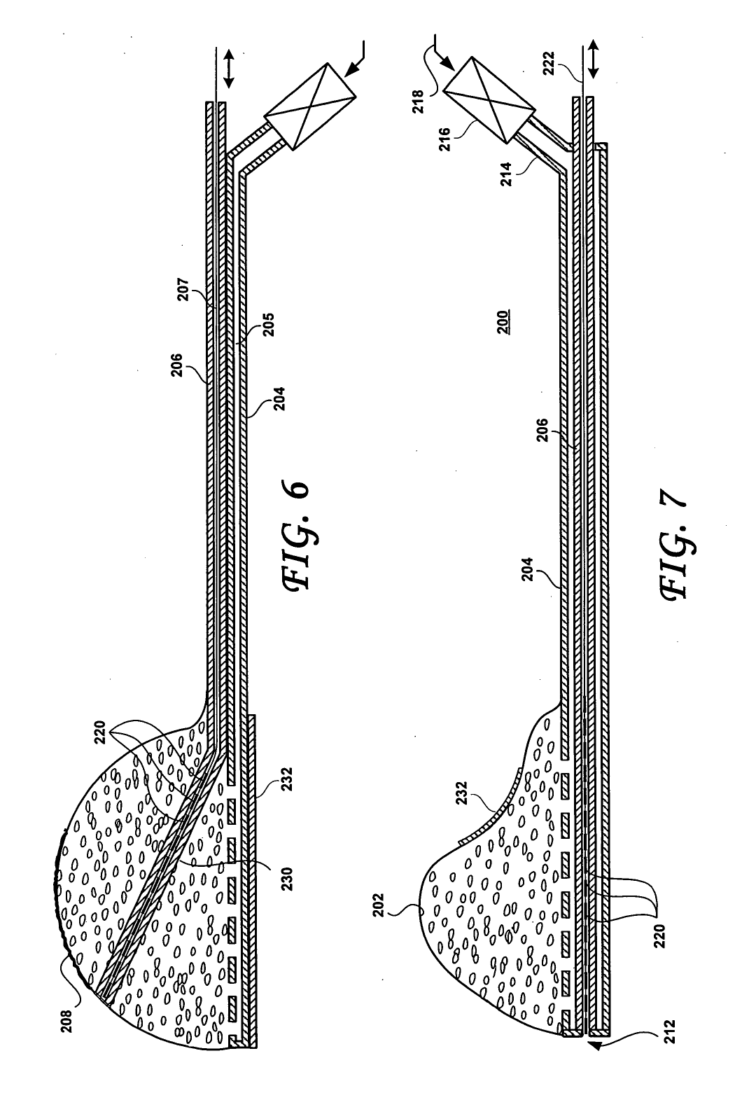

FIG. 2 is a cross-sectional view of a short-term post surgical cavity treatment device 200, according to an embodiment of the present invention, with the inflatable balloon thereof in a non-inflated or partially inflated state. As shown, the device 200 includes an inflatable balloon 202 that is coupled to a first tube 204 that defines an inflation lumen 205 that emerges into the interior of the inflatable balloon 202 by way of a plurality of openings 210 defined within the first tube 204. A gas or liquid 218 (e.g., saline, air) may be introduced into the inflation lumen 205 and into the inflatable balloon 202 through a suitable valve 216, such as a two or three-way stopcock. The device 200 may also include a second tube 206 that defines a treatment lumen 207. The second tube 206 may be located coaxially within the first tube 204 (as best shown in FIG. 4) or the second tube 206 may be located adjacent to the first tube 204, as shown in FIG. 6. The treatment lumen 207 defines an inlet 211 and an outlet 212. In use of the device 200, the outlet 212 emerges within the cavity 126. A semi porous membrane or plug (such as shown at 224 in FIG. 5, for example) may be disposed within the outlet 212 to allow passage of fluids from the treatment lumen 207 to the cavity 126.

As suggested by reference numeral 208, at least a portion of the inflatable balloon 202 may be treated, coated or otherwise configured so as to carry out a beneficial function within the post surgical cavity 126. For example, at least a portion of the outer surface of the inflatable balloon 202 that is, in use, in contact with the interior walls of the cavity 126 may be coated with one or more biologically active substances (such as collagen, gelatin, mucopolysaccarides, hyaluronic acid, for example). Alternately, or in addition, at least a portion of the outer surface of the balloon 202 may be or may include a layer of textured material (such as porous polyurethane, for example) defining various pore densities and/or architectures. At least a portion of the outer surface of the inflatable balloon 202 may be treated with one or more physical methods of surface treatment (such as, for example, plasma or corona discharge surface treatment methods). Alternately, or in addition, at least a portion of the outer surface of the balloon 202 may be chemically treated by chemical methods of treatment (including, for example, surfactants, such as TDMAC—tri-dodecyl methyl—ammonium chloride) with consequent attachment of one or more charged biologically active substances such as antibiotics, specifically cephalosporins and penicillins, for example.

The embodiment of the present short-term cavity treatment device 200 is shown in FIG. 2 in a state in which the balloon 202 is in a deflated or only partially inflated state. FIG. 3 shows the embodiment of the present short-term post surgical cavity treatment device 200 of FIG. 2, in which the balloon 202 is in an inflated state. Numeral 218 is intended to reference a gas, fluid or any other balloon inflation medium. Introduction of the gas or fluid 218 is carried out through the valve 216. According to one embodiment, the valve 216 may be configured to slowly release the gas or fluid 218 at a predetermined rate. In this manner, after all or substantially all of the gas or fluid 218 has been released from the balloon 202, the device may be readily removed from the patient. According to other embodiments, the valve 216 is configured to allow for manual deflation of the balloon 202.

The inflated balloon 202 may be configured to establish or promote hemostasis via physical and/or chemical means. As the balloon 202 is inflated, it applies physical pressure against the interior walls of the cavity 126, and thus promotes hemostasis. Moreover, the coating(s) 208 on at least a portion of the outer surface of the balloon 202 may also promote hemostasis. The coating(s) 208 on the exterior surface of the balloon 202 may also have other beneficial functions, such as antimicrobial prophylaxis using antibiotics and/or antimicrobial substances, for example. Alternately or in addition, the coating(s) 208 may be chosen so as to provide other beneficial actions such as, for example, anti-inflammatory, pain control, steroid therapies and/or anti adhesion therapy, for example.

The balloon 202 may be formed of or include a polymeric material such as, for example, silicone. Any portion of the balloon 202 may be reinforced with one or more additional layers of a reinforcing polymer. Alternatively, the balloon 202 may be formed so as to be locally thicker in such regions thereof wherein it is desired to limit the expansion of the balloon upon inflation. Such locally thicker region(s) and/or reinforcing layer(s) (such as shown at 232 in FIGS. 6 and 7) may influence the ultimate shape that the balloon 202 assumes after inflation. Indeed, the portions of the balloon 202 provided with a reinforcing layer will be relatively less elastic than portions thereof that are not provided with such reinforcing layers 232. Those relatively inelastic portions of the balloon 202 will not expand upon inflation of the balloon 202 to the same extent as those portions of the balloon devoid of such reinforcing layers 232. By judiciously selecting the thickness of the balloon 202 and/or controlling the emplacement and nature of the reinforcing layers 232, the balloon 202 may be made to assume most any shape upon inflation. For example, the balloon 202 may be configured so as to assume a domed or igloo shape upon inflation, as shown in FIG. 7. In this manner, the balloon 202 may be made to assume the shape of the cavity 126. Accordingly, the present short-term post surgical cavity treatment device 200 may be configured with a balloon that inflates to most any desired shape. The present short-term post surgical cavity treatment device 200 may be matched with the excisional device used to create the cavity, so as to match the shape of the inflated balloon thereof with the shape of the cavity created by the excisional device used to create the cavity.

The treatment lumen 207 may be used to inject or otherwise introduce various active substances and/or therapeutic agents into or within the vicinity of the cavity 126. Such active substances may remain within the treatment lumen 207 or may be caused to be introduced within the cavity 126, within the interstitial space between the interior cavity walls and the external surface of the balloon 202. Such active substances and/or therapeutic agents may include, for example, antibiotics, pain medications, steroids, anti adhesion agents. Such active substances and/or therapeutic agents may alternatively (or in addition) include dyes and/or pigments for lymphatic mapping purposes. Moreover, the treatment lumen may be used to provide localized radiation therapy using various isotopes and/or provide localized radiation therapy using other sources of energy (including, for example, an X-ray source). The treatment lumen 207 may also be advantageously used to illuminate a photodynamic substance with, for example, a source of laser light.

As shown in FIG. 3, the treatment lumen 207 may advantageously be used for brachytherapy within the cavity 126 by introducing therein a train of radioactive seeds 220. The radioactive seeds 220 may be coupled to one another by means of a suture material 222 or other wire 222 of suitable material. The train of radioactive seeds 220 may be advanced and retracted within the cavity 126 by alternately pushing and pulling on the wire 222.

FIG. 5 is a cross-sectional view of another embodiment of the present short-term post surgical cavity treatment device, according to the present invention. As shown, the position of the treatment lumen with respect to the center of the cavity 126 and with respect to the center of the space defined by the inflated balloon 202 may be changed. Specifically, the distal portion 230 of the second tube 206 may be bent relative to the proximal portion of the second tube 206. Indeed, the distal portion 230 of the second tube 206 may be disposed so as to traverse the space within the inflated balloon 202. For example, the distal portion 230 of the second tube 206 may be disposed so as to be substantially centered within the balloon 202 when the balloon 202 is inflated. As shown, the outlet 212 (if present) of the treatment lumen 207 may be disposed at a predetermined elevation with respect to the longitudinal axis of the proximal portion of the treatment lumen 207, so as to promote an even distribution of any active and/or therapeutic agent within the cavity 126 and the surrounding tissue. As shown in FIG. 6, the configuration of the distal portion 230 of the second tube 206 (and thus of the distal portion of the treatment lumen 207) may thus be adjusted in order to provide the optimal dose distribution in case of local radiation therapy.

The present short-term cavity treatment device may be introduced into the cavity 126 through the original incision and the access path 127 created by the excisional or biopsy device. According to one embodiment, a simple introducer rod may be inserted within the treatment lumen 207 of the second tube 206. The distal end of the introducer rod would then abut against the semipermeable membrane 224 and enable the physician to push against the introducer to advance the present short-term cavity treatment device into position within the cavity 126. The semi porous membrane 224, therefore, should have sufficient mechanical strength as to withstand the mechanical pressure thereon exerted by the introducer during placement of the device 200 within the cavity 126. The introducer may also have locational and/or prehensile features (visible indicia, wings, etc.) that enable the physician to immediately ascertain the orientation of the balloon 202 within the cavity 126. Alternatively, a trocar coated with friction reducing substance can be used to introduce the present short-term cavity treatment device within the cavity 126.

According to an embodiment of the present invention, the short-term post surgical cavity treatment device includes an expandable balloon that may be formed by dipping a mandrel of a desirable shape and dimensions into a silicone (for example) dispersion. The base of the balloon may be increased, as shown at 232 in FIG. 6 by attaching an additional layer or layers of, for example, a silicone and/or polyurethane film onto the balloon 202. The first and second tubes 204, 206 may be formed of a flexible polymeric material. The inflation lumen of the first tube 204 may be attached to the base of the balloon and may be configured to emerge within the balloon 202. The second tube 206 may be attached to the wall of the balloon 202 and may include a semi permeable membrane 224. The inflation lumen 205 of the first tube 204 may be used to inflate the balloon 202 with normal saline through the two or three way stopcock 216 located on the proximal end of the device 200. The treatment lumen 207 of the second tube 206 may be advantageously be used for the various treatment modalities such as local brachytherapy and for the introduction of biologically active agents. Various dies and pigments for the lymphatic mapping may also be injected through this treatment lumen 207. For example, the surface of the balloon 202 may be coated with a thrombogenic agent such as, for example, collagen or gelatin to induce hemostasis or with hyaloronic acid or a derivative thereof to inhibit or prevent tissue adhesion.

While the foregoing detailed description has described preferred embodiments of the present invention, it is to be understood that the above description is illustrative only and not limiting of the disclosed invention. Those of skill in this art may recognize other alternative embodiments and all such embodiments are deemed to fall within the scope of the present invention.

What is claimed is:

1. A method of treating a surgical cavity of a patient, the surgical cavity defining a narrow access path and a relatively larger cavity chamber, the method comprising the steps of:
providing a cavity treatment device having a balloon and a shaft coupled to the balloon, the balloon being configured to assume a collapsed un-inflated state and an inflated state in which the balloon assumes a shape that defines a narrow proximal portion and a wider distal portion;
inserting the balloon into the surgical cavity while the balloon is in the collapsed un-inflated state;
causing the balloon to assume its inflated state such that the narrow proximal portion substantially fills the narrow access path and the wider distal portion substantially fills the relatively larger chamber.

2. The method of claim 1, wherein the providing step is carried out with the shaft defining an inflation lumen that is in fluid communication with the balloon, and wherein the causing step includes a step of introducing a gas or a fluid into the inflation lumen to inflate the balloon.

3. The method of claim 2, wherein the introducing step is carried out such that an exterior surface of the balloon presses against tissue walls of the surgical cavity so as to provide hemostatis within the surgical cavity.

4. The method of claim 1, wherein the providing step is carried out with the shaft defining a treatment lumen having a distal free end that is configured to be in fluid communication with the surgical cavity, and wherein the method further includes a step of introducing a therapeutic agent into the treatment lumen to deliver the therapeutic agent to the surgical cavity.

5. The method of claim 1, wherein the providing step is carried out with the shaft defining a treatment lumen having a semipermeable membrane disposed at a distal free end thereof, and wherein the method further includes a step of introducing a therapeutic agent into the treatment lumen to deliver the therapeutic agent to the surgical cavity through the semipermeable membrane.

6. The method of claim 1, wherein the providing step is carried out with the shaft defining a treatment lumen that is in fluid communication with the surgical cavity, and wherein the method further includes a step of introducing a lymphatic mapping agent into the surgical cavity through the treatment lumen.

7. The method of claim 1, wherein the providing step is carried out with the shaft defining a treatment lumen that is in fluid communication with the surgical cavity, and wherein the method further includes a step of introducing a radioactive substance into the surgical cavity through the treatment lumen.

8. The method of claim 1, wherein the providing step is carried out with the shaft defining a treatment lumen that is in fluid communication with the surgical cavity, and wherein the method further includes a step of introducing a photodynamic substance into the surgical cavity through the treatment lumen.

9. The method of claim 1, wherein the providing step is carried out with the shaft defining a treatment lumen that is in fluid communication with the surgical cavity, and wherein the method further includes a step of introducing a plurality of radioactive elements into the treatment lumen.

10. The method of claim 1, wherein the providing step is carried out with the shaft defining a treatment lumen and wherein a distal portion of the treatment lumen is configured to bend so as to traverse an interior space defined by the balloon in the inflated state and wherein the method further includes a step of introducing a plurality of radioactive elements into the treatment lumen.

11. The method of claim 1, wherein the providing step is carried out with at least a portion of an exterior surface of the balloon coated with a biologically active substance.

12. The method of claim 11, wherein the biologically active substance in the providing step has at least one of antimicrobial, antibiotic, anti-inflammatory, analgesic, steroidal and anti-adhesion function.

13. The method of claim 11, wherein the biologically active substance in the providing step is at least one of collagen, gelatin, a mucopolysaccaride and hyaluronic acid.

14. The method of claim 1, wherein the providing step is carried out with at least a portion of an exterior surface of the balloon defining a predetermined texture.

15. The method of claim 14, wherein the predetermined texture is achieved by exposing the exterior surface to at least one of a plasma and corona discharge surface treatment.

16. The method of claim 1, wherein the providing step is carried out with at least a portion of an exterior surface of the balloon being chemically treated with a surfactant.

17. The method of claim 1, wherein the providing step is carried out with the balloon having a first portion that is relatively less elastic than a second portion of the balloon.

18. The method of claim 1, wherein the providing step is carried out with the balloon having a first portion that is relatively thicker than a second portion of the balloon.

19. The method of claim 1, wherein the providing step is carried out with the balloon assuming a generally igloo shape when the balloon is in the inflated state.

20. The method of claim 1, wherein the providing step is carried out with the shaft defining an inflation lumen that is in fluid communication with the inflatable balloon and a valve fitted to the inflation lumen, the valve being configured to enable the balloon to gradually deflate at a predetermined rate.

21. The method of claim 20, further including a step of removing the cavity treatment device from the patient when the balloon has deflated and assumed its deflated state.

22. The method of claim 1, wherein the providing step is carried out with the shaft including visible indicia that are configured to enable a physician to correctly orient the balloon within the surgical cavity during the inserting step.

23. A surgical cavity treatment device for treating a surgical cavity having a narrow access path and a relatively larger cavity chamber, the cavity treatment device comprising:
a balloon configured to assume a collapsed un-inflated state and an inflated state;
a shaft coupled to the balloon; and
an inflation lumen that is in fluid communication with the balloon, and
a treatment lumen having an opening that is configured to be in fluid communication with the surgical cavity to enable delivery of a therapeutic agent to the surgical cavity.

24. The surgical cavity treatment device of claim 23, further comprising a semipermeable membrane disposed at the opening in the treatment lumen.

25. The surgical cavity treatment of claim 23, wherein the treatment lumen defines a proximal portion and a distal portion that is substantially co-extensive with the balloon and wherein the distal portion is configured to bend at an angle relative to the proximal portion so as to traverse an interior space defined by the balloon in the inflated state.

26. The surgical cavity treatment device of claim 23, wherein at least a portion of an exterior surface of the balloon is coated with a biologically active substance.

27. The surgical cavity treatment device of claim 26, wherein the biologically active substance has at least one of antimicrobial, antibiotic, anti-inflammatory, analgesic, steroidal and anti-adhesion function.

28. The surgical cavity treatment device of claim 26, wherein the biologically active substance in the providing step is at least one of collagen, gelatin, a mucopolysaccaride and hyaluronic acid.

29. The surgical cavity treatment device of claim 23, wherein at least a portion of an exterior surface of the balloon has a predetermined texture achieved by at least one of a plasma and corona discharge surface treatment.

30. The surgical cavity treatment device of claim 23, wherein at least a portion of an exterior surface of the balloon is chemically treated with a surfactant.

31. The surgical cavity treatment device of claim 23, wherein the balloon has a first portion and a second portion, the first portion being relatively less elastic than the second portion.

32. The surgical cavity treatment device of claim 23, wherein the balloon has a first portion and a second portion, the first portion being relatively thicker than the second portion.

33. The surgical cavity treatment device of claim 23, wherein, in its inflated state, the balloon has a generally igloo shape.

34. The surgical cavity treatment device of claim 23, further comprising a valve fitted to a proximal end of the inflation lumen, the valve being configured to enable the balloon to gradually deflate at a predetermined rate.

35. The surgical cavity treatment device of claim 23, further comprising visible indicia on the shaft, the visible indicia being configured to enable a physician to correctly orient the balloon within the surgical cavity.

36. A method for carrying out brachytherapy in a post surgical cavity within a patient, the method comprising the steps of:
providing a cavity treatment device that includes a balloon configured to assume a collapsed state and an expanded state; a shaft coupled to the balloon;
introducing the balloon of the cavity treatment device into the cavity with the balloon in its collapsed state;
causing the balloon to assume its expanded state, and
inserting and advancing a treatment material into the treatment lumen.

37. The method of claim 36, wherein:
the inserting step is carried out with the treatment material being a radioactive material.

38. The method of claim 36, wherein:
the inserting step is carried out with the treatment material passing through a semipermeable membrane disposed at an opening in the treatment lumen.

39. The method of claim 36, wherein the inserting step is carried out by introducing a lymphatic mapping agent into the surgical cavity through the treatment lumen.

40. The method of claim 36, wherein the inserting step is carried out with the material being a photodynamic substance.

41. The method of claim 36, wherein the inserting step is carried out with the material being a plurality of radioactive elements.

42. The method of claim 36, wherein the providing step is carried out with the treatment lumen configured to bend so as to traverse an interior space defined by the balloon in the inflated state.
